(19) United States
(12) Patent Application Publication
(10) Pub. No.: US 2016/0051629 A1
(43) Pub. Date: Feb. 25, 2016

(54) INHIBITION OF RIP KINASES FOR TREATING LYSOSOMAL STORAGE DISEASES

(71) Applicant: YEDA RESEARCH AND DEVELOPMENT CO. LTD., Rehovot (IL)

(72) Inventors: Anthony FUTERMAN, Rehovot (IL); Einat VITNER, Rehovot (IL); Ran SALOMON, Rehevot (IL)

(21) Appl. No.: 14/781,316

(22) PCT Filed: Apr. 9, 2014
(86) PCT No.: PCT/IL2014/050343

§ 371 Date: Sep. 30, 2015

Related U.S. Application Data
(60) Provisional application No. 61/811,818, filed on Apr. 15, 2013.
(60) Provisional application No. 61/908,118, filed on Nov. 24, 2013.

(51) Int. Cl. A61K 38/20

(52) U.S. Cl. CPC A61K 38/2006

(57) ABSTRACT

The present invention provides compositions and methods for treating lysosomal storage disease characterized by elevation of RIP kinase in a subject in need thereof using at least one RIP kinase inhibitor.

### FIELD OF THE INVENTION

The present invention relates to compositions and methods for treating lysosomal storage diseases in a subject in need thereof. In particular, the present invention relates to the treatment of lysosomal storage diseases by inhibiting the expression or activity of RIP kinase.

### BACKGROUND OF THE INVENTION

Lysosomal Storage Disorders (LSDs) are inherited disorders caused by a defect in lysosomal function that results in accumulation of substances within the lysosome of cells. This defect is a consequence of deficiency of specific enzymes that are normally required for the breakdown of certain complex carbohydrates, and typically a defect in a single enzyme leads to the symptoms of a certain disease. Nearly 50 types and subtypes of LSDs have been identified and taken together they are estimated to affect about 1 in 7,700 births.

Krabbe disease is an inherited, often fatal disorder affecting the central nervous system. The disease affects muscle tone and movement, and may cause vision and hearing loss among other devastating effects. It is caused by the deficiency of an enzyme called galactosylceramidase (GALC; EC 3.2.1.46). This enzyme deficiency impairs the growth and maintenance of myelin. There is no cure for Krabbe disease and treatment mainly involves approaches designed to ease symptoms.

Gaucher disease (GD) is the most common Lysosomal Storage Disorder. This disease is caused by mutations in the Gba gene encoding the lysosomal hydrolase, glucocerebrosidase (GlcCerase; EC 3.2.1.45), which results in accumulation of glucosylceramide (GlcCer). Patients with GD are usually classified into three types, based on the presence or absence of neurological manifestations and their rate of progression. Type 1 (or non-neuropathic type) is the most common form of the disease, occurring in approximately 1 in 50,000 live births. Type 1 patients exhibit a broad spectrum of severity, and some can remain asymptomatic throughout life. Most type 1 patients exhibit enlargement of the spleen and liver, skeletal abnormalities and bone lesions, and sustained inflammatory reactions. Hepatic glucosylceramide levels are elevated from about 20-fold to about 400-fold above normal levels in type 1 Gaucher patients. Type 2 (acute infantile) and type 3 (juvenile or early adult onset) forms comprise only 4% of GD patients. Collectively, type 2 and 3 are referred to as neuropathic (also known as neuronopathic) GD (nGD) since those types display Central Nervous System (CNS) involvement in addition to systemic disease. The main sign is severe difficulty (in type 3), or a total inability (in type 2) to generate saccades (ocular motor apraxia).

Patients with type 2 GD usually die before 3 years of age. Type 2 patients fail to thrive, and display severe and rapidly progressive brainstem degeneration. The most frequent initial clinical signs are hyperextension of the neck, swallowing impairment and strabismus. The most common cause of death is prolonged spontaneous apnea which occurs with increased frequency in the later stages of the disease.

Type 3 patients present similar signs to type 2 patients but with a later onset and decreased severity, and these patients usually survive until adolescence or adulthood. Eye movement abnormalities are common in nGD and their detection is diagnostic of this disorder. In type 3 nGD, oculomotor signs may precede the appearance of overt neurological signs by many years. Auditory brainstem response (ABR) abnormalities are also an early neurological sign in nGD. These symptoms may be isolated, or appear together with developmental delay and seizures.

Current treatment of GD is Enzyme Replacement Therapy (ERT) or Bone Marrow Transplantation (BMT). Specifically, ERT provides good relief of symptoms, but this treatment is lifelong and highly expensive. In addition, ERT does not ameliorate the damage to the CNS that exists in type 2/3 patients because the recombinant enzyme used in ERT does not cross the Blood Brain barrier (BBB). BMT is curative when successful but can be associated with severe morbidity and mortality, and only a small fraction of patients have appropriate histocompatible donors. BMT is specifically described for type 1 Gaucher patients for whom ERT is not an option.

Another strategy for treating GD is substrate reduction therapy. The premise behind this strategy relates to individuals where the amount of substrate exceeds the capacity of the endogenous mutant GlcCeraser enzyme to degrade it. Because reducing glucosylceramide influx will restore the balance between substrate synthesis and degradation in the lysosome, inhibition of glucosylceramide biosynthesis may improve the clinical course of the disease. This strategy also is not applicable for type 2 and 3. In particular this strategy has been approved for use in patients with mild to moderate type 1 GD for whom enzyme replacement therapy is not a feasible option.

Yet another possible strategy to treat GD is gene therapy mediated by adenovirus and lentivirus vectors, although significant hurdles still exist with the implementation of gene therapy as a practical and safe therapeutic strategy.

U.S. Pat. No. 7,429,460 to some of the inventors of the present invention discloses that accumulation of glucosylceramide (GlcCer) directly activates the rate-limiting enzyme of phosphatidylcholine (PC) synthesis, CTP:phosphocholine cytidylyltransferase (CCT). Based on this phenomenon the patent discloses methods for screening for compounds that inhibit the synthesis of phosphatidylcholine (PC), wherein PC synthesis is increased due to the activation of CTP:phosphocholine cytidylyltransferase (CCT) upon GlcCer accumulation, particularly compounds inhibiting CCT.

Very little is known about the neuropathological events that cause neurological abnormalities associated with nGD. Previous systematic neuropathological analysis of autopsy human GD brains highlighted specific patterns of astrogliosis and neuron loss, in addition to non-specific gray and white matter gliosis (Wong, K. et al., Mol. Genet. Metab., 82:192-207; 2004). A recent study conducted by some of the inventors of the present invention elucidated the onset and progression of various neuropathological changes (including microglial activation, astrogliosis and neuron loss) in a mouse model of nGD, and documented the brain areas that are first affected during the course of the disease. In addition, it was established that microglial activation and astrogliosis are spatially and temporally correlated with selective neuron loss (Farfel-Becker, T. et al., Hum. Mol. Genet., 20:1375-1386; 2011).

Further study by some of the inventors of the present invention suggested the involvement of cathepsins in the neuropathology of neuronal forms of GD (Vitner, E. B. et al., Hum. Mol. Genet., 19:3583-3590; 2010).

Finally, a more recent study conducted by some of the inventors of the present invention delineated the role of neuroinflammation in the pathogenesis of neuronopathic Gaucher's disease and show significant changes in levels of inflammatory mediators in the brain of a neuronopathic Gaucher's disease mouse model. BBB disruption was also evident in mice with neuronopathic Gaucher's disease. Finally, extensive elevation of nitrotyrosine, a hallmark of peroxynitrite (ONOO—) formation, was observed and demonstrated to be consistent with oxidative damage caused by macrophage/microglia activation. Namely, the observed results suggested a cytotoxic role for activated microglia in neuronopathic Gaucher's disease (Vitner E B, Brain. 2012 June; 135(Pt 6):1724-35.

Taken together, current therapies possess several drawbacks in treating type 1 GD. In addition, as of to date, there is no effective therapeutic approach for type 2/3 GD.

A paper of the inventors of the present invention, published after the priority date, describes that modulating the receptor-interacting protein kinase 3 (RIP3) pathway improved neurological and visceral disease in a mouse model of GD. It was demonstrated that Rip3 deficiency improved the clinical course of GD mice with increased survival, motor coordination and salutary effects on cerebral as well as hepatic injury (Vinter E B et al., Nat Med. February; 20(2):204-208; 2014. doi: 10.1038/nm.3449. Epub 2014 Jan. 19).

There is a need for means and methods for treating lysosomal storage disorders including Gaucher's disease that provide patients with a higher quality of life and achieve a better clinical outcome.

### SUMMARY OF THE INVENTION

The present invention is related to the field of lysosomal storage diseases. In particular, the present invention provides methods for the treatment of Gaucher's disease, including of severe neuronopathic types of the disease, or Krabbe's disease.

The present invention is based in part on the unexpected discovery that mice deficient of the Receptor-Interacting Protein (RIP) kinase, particularly RIP3 kinase were significantly less sensitive to the GlcCerase inhibitor conduritol β epoxide (CBE) in comparison to RIP3 expressing mice that exhibited symptoms of Gaucher disease upon administration of CBE. The RIP3 deficient mice showed significant increase in life span and in motor coordination abilities. In addition, elevated amounts of RIPs were found also in a mouse model of galactosylceramide lipidosis, also known as Krabbe disease.

Thus, according to one aspect, the present invention provides a method of treating a subject affected with a lysosomal storage disease characterized by elevation of RIP kinase, the method comprising administering to the subject a therapeutically effective amount of at least one Receptor-Interacting Protein (RIP) kinase inhibitor, thereby treating the lysosomal storage disease.

According to certain embodiments, the lysosomal storage disease is selected from the group consisting of Gaucher's disease and Krabbe disease.

According to certain embodiments, the lysosomal storage disease is Gaucher's disease.

Unexpectedly, inhibiting the expression of RIP3 is effective in ameliorating the symptoms of Gaucher's disease not only in the neuronopathic form of the disease but also in the less severe types. Thus, according to certain embodiments, the method of the present invention is useful in treating Type 1 Gaucher's disease, Type 2 Gaucher's disease or Type 3 Gaucher's disease. Each possibility represents a separate embodiment of the present invention.

According to other embodiments, the lysosomal storage disease is Krabbe.

According to certain embodiments, the at least one RIP kinase inhibitor is capable of inhibiting RIP activity or expression. As used herein the term “inhibiting RIP activity” includes, but is not limited to, impairing the functionality of RIP and inducing RIP's degradation. According to certain typical embodiments, the RIP kinase inhibitor is a small molecule compound capable of inhibiting the RIP kinase activity. According to certain embodiments, the small molecule is necrostatin-1.

According to certain embodiments, the RIP kinase inhibitor is in a form capable of passing the blood brain barrier (BBB).

Various types of RIP kinases are known in the art. According to certain typical embodiments of the present invention, the inhibited RIP kinase is selected from the group consisting of RIP1 and RIP3. Each possibility represents a separate embodiment of the present invention. According to one embodiment, the inhibited RIP kinase is human RIP1, having the accession number NP_{—}003795.2. According to another embodiment, the inhibited RIP kinase is human RIP3, having the accession number RIP3: NP_{—}006862.2.

According to certain embodiments, the compound inhibiting RIP1 and/or RIP3 has specificity or some selectivity to inhibition of RIP1, RIP3 or both.

Any compound known in the art to be capable of inhibiting RIP kinase expression or activity as well as those to be further found are encompassed within the scope of the present invention.

According to certain embodiments, the subject is a human.

According to certain embodiments, the method further comprises administering to the subject an effective amount of at least one additional therapeutically active compound. According to certain embodiments, the additional therapeutic compound is a compound known to be effective in treating a lysosomal storage disease, particularly Gaucher's disease or Krabbe disease.

According to some embodiment, the additional active compound is IL-1β receptor antagonist. According to exemplary embodiments, the IL-1β receptor antagonist is Anakinra.

In some embodiments said subject in need is selected from the group consisting of: a patient afflicted with said disease or disorder, a patient afflicted with said disease or disorder wherein said patient is in remission, a patient afflicted with said disease or disorder having manifested symptoms associated with said disease or disorder, and any combination thereof.

The RIP inhibitor compounds or pharmaceutical composition comprising same can be administered to the subject via any suitable route as is known to a person skilled in the art.

In certain embodiments, the active compound or pharmaceutical composition comprising same is administered orally or parenterally. In some embodiments, the route of administration is selected from the group consisting of: intravenously, subcutaneously, intra-arterially, intraperitoneally, ophthalmically, intramuscularly, buccally, rectally, vaginally, intraorbitally, intracerebrally, intradermally, intracranially, intraspinally, intraventricularly, intrathecally, intracisternally, intracapsularly, intrapulmonarily, intranasally, transmucosally, transdermally, inhalation, and any combination thereof. Each possibility is a separate embodiment of the invention.

According to another aspect, the present invention provides a pharmaceutical composition comprising a pharmaceutically effective amount of a RIP kinase inhibitor and a pharmaceutically acceptable carrier for use in the treatment of a lysosomal storage disease characterized by elevation of RIP kinase.

The RIP kinase type, RIP kinase inhibitor and the lysosomal storage disease are as described hereinabove.

According to certain embodiments, the pharmaceutical composition further comprises at least one additional therapeutically active compound. According to some embodiments, the additional active compound is IL-1 receptor antagonist. According to exemplary embodiments, the IL-1 receptor antagonist is Anakinra. According to certain embodiments, the additional therapeutic compound is a compound known to be effective in treating a lysosomal storage disease, particularly Gaucher's disease or Krabbe disease.

The RIP inhibitor compounds or pharmaceutical composition comprising same can be administered to the subject via any suitable route as is known to a person skilled in the art.

According to yet additional aspect, the present invention provides a pharmaceutical composition comprising at least one RIP kinase inhibitor and a pharmaceutically acceptable carrier, excipient or diluent.

The RIP kinase types and RIP kinase inhibitor are as described hereinabove.

According to yet additional aspect, the present invention provides a kit for the treatment of a subject affected with lysosomal storage disease characterized by elevation of RIP kinase, the kit comprising a container containing a pharmaceutical composition comprising at least one RIP kinase inhibitor and a pharmaceutically acceptable carrier, excipient or diluent; and written instructions for use of said pharmaceutical composition.

According to certain embodiments, the lysosomal storage disease is selected from the group consisting of Gaucher and Krabbe.

### BRIEF DESCRIPTION OF THE DRAWINGS

FIG. 1 shows that neuronal cell death in GD mice is non-apoptotic. Data were obtained from Gba^{flox;flox}; Nestin-Cre mice (−/−) and their respective littermate controls (+/−), or from C57BL/6 mice that were injected i.p. with either 50 mg/kg/day CBE or phosphate-buffered saline (PBS) starting at 8 days of age.

FIG. 1A: Fluoro-Jade C staining indicating dying neurons in cortical layer V of 21 day-old −/− mice (n=5) and of 27 day-old CBE-treated mice (n=2); for CBE-treated mice, a further 6 biological replicates were performed in other mouse strains using different doses of CBE, with similar results. Bar—100 μm. No staining was observed in control brains.

FIG. 1B: TUNEL staining of cortical layer V in 21 day-old −/− mice (n=3) and in 27 day-old CBE-treated (n=2) mice. Sections were counterstained with DAPI. Only DAPI staining has been detected. Bar—100 μm.

FIG. 1C: Activity of caspases 8, 9, and 3/7 in cortical homogenates of 16 (upper panel) and 21 (middle panel) day-old −/− mice and in 27 day-old CBE-treated mice (lower panel). Activities were normalized to 100% of the values of control mice. Values are average±s.e.m. (n=2 for CBE-treated mice, n=7 for 21 day-old −/− mice and n=5 for all others). *p<0.05.

FIG. 1D: Western blots of caspase 8 in homogenates (100 μg protein) from the brains of 27 day-old CBE-treated mice (n=2) and of 21 day-old −/− mice (n=4). GAPDH served as a loading control. A liver homogenate from mice treated with a Fas-activating antibody (Jo2) acted as a control for caspase 8 cleavage.

FIG. 1E: Western blots of the cleavage product of PARP (Mr of 89 kDa) in homogenates (100 μg protein) from brains of 27 day-old CBE-treated mice (n=2) and of 21 day-old −/− mice (n=4). GAPDH served as a loading control. A liver homogenate from mice treated with a Fas-activating antibody (Jo2) acted as a control for PARP cleavage.

FIG. 1F: Real-time PCR of cFLIPL and cFLIPS in cortical homogenates from 21 day-old CBE-treated mice (n=3) and from 16 and 21 day-old −/− mice (n=4). Results are expressed as fold-change versus untreated or +/− mice, and are average±s.e.m. CT values were normalized to levels of TBP. *p<0.05, **p<0.01, ***p<0.001, between −/− versus +/− mice and between mice treated with CBE versus PBS.

FIG. 1G: Western blots of cFLIPL and cFLIPS in homogenates (150 μg protein) from brains of 21 day-old −/− mice (n=4) and from 21 day-old CBE-treated mice (n=5). GAPDH served as a loading control.

FIG. 2 demonstrates elevation of RIP1 and RIP3 in brains from neuronopathic Gaucher disease and Krabbe mice. For figures a-e, data were obtained from 21 day-old Gba^{flox/flox}; Nestin-Cre mice (−/−) and their respective littermate controls (+/−).

FIG. 2A: Real-time PCR of Rip1 and Rip3 in cortical homogenates from 21 day-old −/− mice. Results are expressed as fold-change and are average±s.e.m. (n=3 for +/− mice and n=6 for −/− mice). CT values were normalized to levels of TBP. *p<0.001.

FIG. 2B: Western blot of homogenates (150 μg of protein) from the brains of 16 and 21 day-old −/− mice (n=5). Full-length RIP1 and cleaved RIP1 are indicated by arrows, and unidentified cleavage products of RIP1 are indicated by asterisks. Mr markers are shown. GAPDH was used as loading control.

FIG. 2C: Western blot of homogenates (150 μg of protein) from the brains of 16 and 21 day-old −/− mice. Full-length RIP3 is indicated by an arrow and an unidentified band is indicated by an asterisk. A homogenate from the brain of a Rip3 null (Rip3−/−) mouse was used as a control; note that the upper band, indicated by the arrow, is absent in the Rip3 mill mouse confirming the identity of this band in the Gaucher mice. Mr markers are shown. GAPDH was used as loading control.

FIG. 2D: Double immunofluorescence of brain cells of 16 day-old −/− mice using either anti-RIP3 and anti-Mac-2 (upper panel), or anti-RIP3 and anti-GFAP (lower panel) antibodies. Areas of overlap are indicated in the right-hand panels. Scale bar, 10 μm. Results are representative of three biological replicates.

FIG. 2E: Double immunofluorescence of 16 day-old +/− (control, upper panel) and −/− mice (lower panel) using DAPI, anti-NeuN and anti-RIP3 antibodies; areas of overlap are indicated in the right-hand panels (merged). Arrows indicate nuclear staining of RIP3. Scale bar, 10 μm. Results are representative of three biological replicates.

FIG. 2F: Western blot of homogenates (150 μg of protein) from the brains of 5 week-old Krabbe disease (Twitcher) mice. Blots were probed with anti-RIP1 or anti-RIP3 antibodies. Full-length RIP1, cleaved RIP1 and RIP3 bands are indicated by arrows, and unidentified bands on the RIP1 and RIP3 blots are indicated by asterisks (*). Results are representative of two biological replicates. A homogenate from the brain of a Rip3 null (Rip3−/−) mouse was used as a control and GAPDH as a loading control.

FIG. 2G: Real-time PCR of Rip1 and Rip3 in cortical homogenates from 28 day-old Rip3+/− (control) and Rip3−/− mice treated with either CBE (25 mg/kg/day) or PBS from 8 days of age. Results are expressed as fold-change and are average±s.e.m. (n=3). CT values were normalized to levels of TBP. *p<0.05, **p<0.01, between Rip3+/− and Rip3−/− mice treated with CBE versus PBS.

FIG. 2H: Western blot of homogenates (150 μg of protein) from the brains of 28 day-old Rip3+/− (+/−) and Rip3−/− (−/−) mice treated with either CBE (25 mg/kg/day) or with PBS from 8 days of age. Full-length RIP1 and cleaved RIP1 are indicated by arrows. Full-length RIP3 is indicated by an arrow and an unidentified band is indicated by an asterisk (lower panel). Results are representative of three biological replicates. Mr markers are shown. GAPDH was used as loading control.

FIG. 2I: Real-time PCR of cFLIPL and cFLIPS in cortical homogenates from 28 day-old Rip3+/− (control) and Rip3−/− mice treated with either CBE (25 mg/kg/day) or with PBS from 8 days of age. Results are expressed as fold-change and are average±s.e.m. (n=3). CT values were normalized to levels of TBP. *p<0.05 between Rip3+/− and Rip3−/− mice treated with CBE versus PBS.

FIG. 3 demonstrates that Rip3 deficiency improves the clinical course of Gaucher disease mice. Mice were injected i.p. with 25 mg/kg/day CBE or with PBS from 8 days of age.

FIG. 3A: Body weight of Rip3+/− (control, n=15) and Rip3−/− mice (n=18) treated with either CBE or PBS (n=9 for Rip3+/− and n=7 for Rip3−/− mice). Results are average±s.e.m. *p<0.05, **p<0.01, ***p<0.001 between Rip3+/− and Rip3−/− mice treated with CBE.

FIG. 3B: Rotarod performance of 31 day-old Rip3+/− and Rip3−/− mice. n=5 for PBS-treated mice, n=6 for Rip3+/− mice treated with CBE and n=10 for Rip3−/− mice treated with CBE; results are average±s.e.m. *p<0.001.

FIG. 3C: Kaplan-Meyer survival curves for Rip3+/− (n=5) and Rip3−/− (n=6) mice. Rip3 deficiency significantly extends the lifespan of the mice (p<0.001).

FIG. 3D: Microglial activation (Mac-2) in coronal sections of 28 day-old Rip3+/− and Rip3−/− mice treated with CBE from 8 days of age. Results are representative of three biological replicates. Bar, 250 μm. No staining was observed in control brains.

FIG. 3E: Immunohistochemical staining for the macrophage marker, CD68 in liver sections of 28 day-old Rip3+/− and Rip3−/− mice treated with CBE. Results are representative of three biological replicates. Bar, 40 μm.

FIG. 3F: Serum ALT in 28 day-old Rip3+/− and Rip3−/− mice treated with CBE or PBS (n=3). Results are average±S.D. *p<0.05.

FIG. 3G: Spleen weight of 28 day-old Rip3+/− and Rip3−/− mice treated with CBE (25 mg/kg/day, n=5 for Rip3+/− and n=7 for Rip3−/− mice) or PBS (n=3) for 20 days. Results are average±s.e.m. *p<0.05.

FIG. 4 shows that the clinical course of Gaucher disease mice is TNFα-independent.

FIG. 4A: Body weight of Tnfα +/− (control, n=4) and Tnfα −/− (n=3) mice treated with either CBE (50 mg/kg/day) or PBS (n=3 for Tnfα +/− and n=4 for Tnfα −/− mice) from 8 days of age. Results are average±s.e.m.

FIGS. 4B and 4C: Western blot of homogenates (150 μg of protein) from the brains of 22 day-old Tnfα +/− (+/−) and Tnfα −/− (−/−) mice treated with either CBE (50 mg/kg/day) or PBS from 8 days of age. Full-length RIP1 and cleaved RIP1 are indicated by arrows, and unidentified cleavage products of RIP1 are indicated by asterisks.

FIG. 4B: Full-length RIP3 is indicated by an arrow and an unidentified band is indicated by an asterisk.

FIG. 4C: Results are representative of three biological replicates. Mr markers are shown. GAPDH was used as loading control.

FIG. 5 shows elevation of pro caspase-1 in brains from neuropathic Gaucher disease mice. Western blot of homogenates (150 μg of protein) from the brains of 21 day-old from Gba^{flox/flox}; Nestin-Cre mice (−/−) and their respective littermate controls (+/−).

FIG. 6 shows elevation of pro IL-1β in brains from neuropathic Gaucher disease mice. Western blot of homogenates (150 μg of protein) from the brains of 21 day-old from Gba^{flox/flox}; Nestin-Cre mice (−/−) and their respective littermate controls (+/−).

FIG. 7 shows that IL-1Ra (Anakinra) treatment improves the clinical course of Gaucher disease mice. Mice were injected i.p. with 50 mg/kg/day CBE or CBE+IL-1Ra (Anakinra, 75 mg/kg) from 8 days of age. Body weight (FIG. 7A) and life span (FIG. 7B) of mice treated with either CBE only or CBE+Anakinara (n=3).

### DETAILED DESCRIPTION OF THE INVENTION

The invention is directed to means and methods for treating a subject diagnosed to be at risk of developing or afflicted with a lysosomal storage disease characterized by elevation of RIP3 expression or activity. The method of the invention comprises administering to the subject a therapeutically effective amount of at least one compound effective in inhibiting the expression or activity of Receptor-Interacting Protein (RIP) kinase. According to certain embodiments, the lysosomal storage disease is selected from the group consisting of Gaucher's disease (GD) and Krabbe disease. According to certain exemplary embodiments, the lysosomal storage disease is GD. The invention is applicable to all three types of GD.

The treatments available to date for lysosomal storage diseases resulting from an enzyme deficiency, including type 1 GD are highly expensive and not always effective. As for type 2 and type 3 GD, no efficient therapeutic approach for treating those conditions exists. Very little is known about the biochemical pathways that lead to the nGD pathological events and the mode of neuronal death (i.e., apoptosis versus necrosis) is unidentified. Identification of the mechanisms that underlie neuronal death is critical to developing new treatment strategies for nGD. The inventors of the present invention herein show, for the first time, an elevation in the expression and cleavage levels of RIP1 and/or an elevation in the expression of RIP3 in mice afflicted with GD. In addition, the present invention now shows that mice demonstrating GD having deficient RIP3 expression presented improved clinical manifestation in brain as well as in liver, resulting in extended life span. The present invention further demonstrates elevation of RIP1 and/or RIP3 in model mice of Krabbe disease, which suggests that these enzymes play a key role in additional lysosomal diseases. Therefore, compounds capable of inhibiting the expression and/or activity of RIP1 and/or RIP3 are suitable for the treatment of said diseases. Alteration in RIP1 or RIP3 level was not observed in model mice of the LSDs Niemann Pick type C1, GM1 gangliosidosis and Sandhoff diseases. However, this may be due to not fully matched mice models and/or specific experimental conditions that may mask RIP1 or RIP3 elevation. The present invention is meant to encompass treatment of any LSD characterized by elevation of RIP kinase.

### DEFINITIONS

The terms “RIP” and “RIP kinase” are used herein interchangeably. These terms are further interchangeable with any alternative name or synonyms of this protein known in the art including, but not limited to: RIPK, Cell death protein RIP, Cell death protein RIP kinase, Receptor-interacting protein 1 and Receptor-interacting protein 1 kinase. According to certain embodiments, the RIP kinase is selected from RIP1 and RIP3. According to other embodiments, RIP is RIP1. According to another embodiment, RIP is RIP3.

The terms “lysosomal storage disease” and “LSD” throughout the description and according to the present invention refer to lysosomal storage disease which is characterized by elevation of RIP kinase.

As used herein, the terms “elevation of RIP kinase” and “elevated RIP kinase” are used herein interchangeably and refers to elevation in the expression level and or in the activity of the RIP kinase enzyme.

The terms “Krabbe disease” (KRAH-buh disease) and “Gaucher's disease” (also referred to herein as “Gaucher”, “Gaucher disease” and “GD”) are used herein in their broadest scope as is known in the art and as described in details hereinabove.

The term “affected with” or “afflicted with” are used herein interchangeably and refer to subjects that are carriers of the disease, regardless of the degree of symptom manifestation. The affected subjects can be at any disease phase, including, but not limited to, before burst, at burst, during a continuous course of the disease and after remission.

Accordingly, “a patient affected with lysosomal storage disease” refers to a patient with lysosomal storage disease showing to symptoms, a patient with lysosomal storage disease being patient in remission, a patient with lysosomal storage disease with manifested lysosomal storage disease symptoms and a patient susceptible to lysosomal storage disease. Each possibility is a separate embodiment of the invention.

According to certain embodiments the above mentioned lysosomal disease is Gaucher. Thus, “remission in Gaucher disease” refers to reduction or absence of clinical signs of the disease and a patient “susceptible to Gaucher” refers to a subject having genetic makeup which enhances the chance of the subject to show symptoms of GD.

The term “treating” as used herein, includes, but is not limited to any one or more of the following: abrogating, ameliorating, inhibiting, attenuating, blocking, suppressing, reducing, delaying, halting, alleviating or preventing the symptoms associated with lysosomal storage disease.

Thus, by treating the symptoms associated with lysosomal storage disease it is meant that at least one of the symptoms mentioned herein or is known in the art to be associated with lysosomal storage disease is improved by the method of the present invention.

The present invention provides, for the first time, a method of treating a lysosomal storage disease characterized by elevation of RIP kinase, comprising administering a therapeutically effective amount of a RIP kinase inhibitor, capable of inhibiting RIP expression or activity, to subjects in need of such treatment.

Receptor-interacting protein (RIP) kinases are a group of threonine/serine protein kinases with a relatively conserved kinase domain but distinct non-kinase regions. In humans, five different RIP kinase forms are known, designated RIP1, RIP2, RIP3, RIP4, and RIPS. A number of different domain structures, such as death domain and caspase activation and recruitment domain (CARD), were found in different RIP family members, and these domains have been considered as key feature in determining the specific function of each RIP kinase. It is known that RIP kinases participate in different biological processes, including those in innate immunity, but their downstream substrates are largely unknown. Recent evidence has shown that the signaling pathway of necroptosis, a programmed form of necrosis, depends on the activation of RIP1 and RIP3 in response to death receptors induction. Direct cleavage of the RIPs by caspases prevents necroptotic cell death and it is associated with apoptotic cell death. It was recently shown that RIP1 and RIP3, in addition to their role in necroptosis, contribute to inflammation by activation of the NLRP3 inflammasome in dendritic cells (Kang, T. B. et al., Immunity; 38:27-40; 2013).

Some of the inventors of the present invention have previously shown that nGD is associated with massive neuronal loss in the brains of nGD mouse model (Farfel-Becker, T. et al. 2011, ibid).

The present invention now shows that, unexpectedly, RIP kinases are directly involved in the pathway of the pathological events which induce the relentless neuroinflammatory changes and tissue injury that are characteristic of severe forms of GD. Moreover, it appears that RIP1 and RIP3 are also implicated in the acute neuropathological changes that occur throughout the CNS in Krabbe disease. Without wishing to be bound by any specific theory or mechanism of action, RIP3 elevation in microglia of neuropathic GD mice, together with the improvement in symptoms prior to neuronal loss and the attenuation of the pathological injury in peripheral organs in RIP-deficient mice (Rip3−/− GD mice), support the finding of the present invention that RIP3 is not only a key activator of necrotic cell death, but also orchestrates inflammatory engagement, independent of necrosis. In agreement with this proposed mechanism, elevated expression levels of both pro caspase-1 and pro IL-1β were found in brains of neuropathic GD mice (Gba^{flox/flox}; Nestin-Cre mice (−/−)). Moreover, treatment of GD mice with IL-1β receptor antagonist (Anakinra) improved the clinical course of Gaucher disease in these mice, resulting in better gain weight and life-span parameters. Thus, according to certain embodiments, the methods of the present invention comprise administering to the subject in need thereof RIP kinase inhibitor and at least one additional active compound. According to certain embodiments, the active compound is IL-1 receptor antagonist. According to some exemplary embodiments, the IL-1 receptor antagonist is Anakinra (a recombinant, non-glycosylated version of human IL-1 receptor antagonist, sold under the trade name “KINERET®” (Amgen)).

According to certain embodiments, the present invention provides a method of treating a subject affected with a Gaucher disease, the method comprising administering to the subject a therapeutically effective amount of at least one IL-1β receptor antagonist, thereby treating the Gaucher disease. According to exemplary embodiments, the IL-1β receptor antagonist is Anakinra. According to other embodiments, the present invention provides a pharmaceutical composition comprising a pharmaceutically effective amount of at least one IL-1β receptor antagonist and a pharmaceutically acceptable carrier for use in the treatment of a Gaucher disease.

An association between the presence of the mutation in the Gba gene underlying type 1 GD and Parkinson's disease was shown, with a considerable population of Parkinson's patients having this mutation (Sidransky, M. D, N Engl J Med. 2009 Oct. 22; 361(17): 1651-1661). Together with observation of several subclinical neurological manifestations in type 1 GD patients, it has been suggested that type I GD should be considered a continuum of additional clinical manifestations, exhibiting some neurological abnormalities and susceptible to Parkinson rather than distinct subtype. Thus, mice carrying Gba point mutations or mice treated with lower dose of CBE or mice less sensitive to CBE, may be used further for studying additional diseases, including Parkinson. The amelioration of GD symptoms in RIP deficient mice suggests that RIP inhibitors may be effective in treating Parkinson disease as well. Thus, according to certain embodiment, the RIP inhibitor is for treating Gba mutation-related Parkinson. According to certain embodiments, the present invention provides a method of treating a subject affected with Parkinson's disease comprising administering to the subject a therapeutically effective amount of at least one Receptor-Interacting Protein (RIP) kinase inhibitor, thereby treating Parkinson's disease.

According to the teachings of the present invention, a method of “treating lysosomal storage disease” includes, but is not limited to, administration a compound inhibiting the expression and/or activity of RIP kinase to a subject in order to cure or to prolong the health or survival of said subject beyond that expected in the absence of such treatment.

Hitherto, several attempts to identify inhibitors of the RIP kinases have been reported. For example, the RIP1 kinase inhibitor, necrostatin-1 (Nec-1), has been used to demonstrate the importance of RIP1 in mediating acute tissue injury (for example, Chavez-Valdez, R. et al. 2012. Neuroscience 219, 192-203). Nec-1 crosses the blood-brain barrier and has a half-life of ˜1 h. RIP3 inhibitors (Kaiser, W. J. et al. 2013. J. Biol. Chem. doi:10.1074/jbc.M113.462341), and an inhibitor of necrosis downstream to RIP3 (Sun, L. et al. 2012. Cell 148, 213-227), have demonstrable effects on mouse and human cell lines. They have not been shown, to date, to display efficacy in vivo or to cross the blood brain barrier, however a continuous effort has been taken forward improving these inhibitors and screening for others (Kaiser et al. 2013. ibid).

According to other embodiments, the expression “inhibiting RIP activity” comprises any one or more of the following: attenuating, reducing or preventing necroptosis and/or inflammation associated with RIP kinase expression and/or activity in a cell.

According to yet additional embodiments, inhibiting RIP activity is mediated by at least one or more of: reducing, inhibiting or preventing the expression of RIP, neutralizing the functionality of RIP and inducing RIP's degradation. According to certain embodiments, inhibiting RIP activity is mediated by reducing, inhibiting or preventing the expression of RIP. Inhibiting RIP activity may be mediated directly by interacting with RIP protein, gene or mRNA or indirectly by interacting with a protein, gene or mRNA associated with RIP-mediated activity or expression. According to yet another embodiment, expression is over-expression.

As used herein the term “expression” refers to the production of a functional end-product e.g., an mRNA or a protein of a gene in a cell. As used herein the term “over-expression” is an expression of a gene above the expression level of that gene under normal conditions. By “normal conditions” it is meant a steady state condition wherein no pathological condition associated with GD occurs and/or no medical intervention is required.

According to certain embodiments, a reduction in RIP expression or activity comprises a reduction of about 10%, 20%, 30%, 40%, 50%, 60%, 70%, 80%, 90%, or more, as compared to the expression or activity of the RIP enzyme under the same conditions in the absence of a RIP inhibitor. Each possibility represents a separate embodiment of the present invention.

As used herein the term “RIP inhibitor” refers to an agent or compound capable of inhibiting RIP activity and/or expression and derivatives or salts thereof. According to yet other embodiments, the RIP inhibitor is selected from the group consisting of: a chemical agent or moiety, a protein, a polypeptide or a peptide, and a polynucleotide molecule. Each possibility represents a separate embodiment of the invention. The scope of the present invention encompasses homologs, analogs, variants and derivative of RIP inhibitor, with the stipulation that these variants and/or modifications must inhibit RIP expression and/or activity. According to certain embodiments, the RIP kinase inhibitor is capable of passing through the blood brain barrier (BBB) or is formulated to pass through the BBB.

One or more of the RIP inhibitors of the invention may be present as a salt. The term “salt” encompasses salts formed by standard acid-base reactions between an acidic moiety and an organic or inorganic cation. The term “organic or inorganic cation” refers to counter-ions for the carboxylate anion of a carboxylate salt or the counter ion for the phenoxide moiety. The counter-ions are chosen from the alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylainmonium, 2 hydroxyethylainmonium, bis(2-hydroxyethyl)ammonium, phenylethylbenzylammonium, dibenzylethylenediammonium, and like cations.

According to certain exemplary embodiments, the RIP kinase inhibitor is a small molecule capable of inhibiting the activity of RIP kinase protein. Any small molecule known to have such activity can be used according to the teachings of the present invention. According to further typical embodiments, the small molecule may be formulated within a pharmaceutical composition. According to certain embodiments, the small molecule is capable of passing through the blood brain barrier (BBB) or is formulated to pass through the BBB.

Methods of screening for new RIP inhibitor compounds and identification of preferred inhibitors are as known in the art. For example, a screening method may comprise a cell culture wherein the cells are over expressing RIP. The cell culture is then exposed to at least one candidate inhibitory compound and the accumulation of a downstream substrate or enzyme is measured. RIP3 enhances assembly and function of the inflammasome and an expected outcome of RIP3 inhibition is a reduction of IL-1β cleavage. Compounds having an effective inhibitory activity are selected.

In other embodiments, RIP3 inhibitor compounds affect the expression of RIP3. In these exemplary embodiments, a screening for reduction of the RIP3 accumulation is implied. For example, since RIP3 is involved in necroptotic cell death, an assay for detecting its inhibition may include the induction of necroptosis on cell and testing the degree of cell death with the compound of interest as described in Sun et al. (Sun L et al, 2012, Cell 148(1-2):213-227). Briefly, on day one, 2,000 HT-29 cells are split into each well of a 384-well assay plate. On day two, necrosis is induced by adding final concentrations of 20 ng/ml TNF-α, 100 nM Smac mimetic, and 20 μM z-VAD to the well. Concurrently, individual compounds from a chemical library of ˜200,000 compounds are delivered into each well at a final concentration of 10 μM. Cell viability in this and subsequent panels are determined by measuring ATP levels by Cell Titer-Glo assay after 24 hrs.

According to yet other embodiments, the RIP inhibitor is a polynucleotide molecule. According to certain embodiments, the polynucleotide molecule is a nucleic acid sequence or a molecule capable of hybridizing to nucleic acids encoding or controlling RIP expression. Exemplary nucleic acid sequences suitable in the context of the present invention include, but are not limited to, an RNA inhibiting (RNAi) molecule, an antisense molecule and a ribozyme. Each possibility represents a separate embodiment of the invention. As used herein, the term RNAi describes a short RNA sequence capable of regulating the expression of target genes by binding to complementary sites in the target gene transcripts to cause translational repression or transcript degradation.

As used herein, the term “RNAi” is a generic term used throughout the specification to include small interfering RNAs (siRNAs), hairpin RNAs, and other RNA species that can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts that can produce siRNAs in vivo.

The designing of a suitable sense and antisense that will serve as RNAi against a target gene is within the knowledge of the skilled in the art. According to certain embodiments the RNAi sequence comprises a sequence complementary to RIP3 polynucleotide. According to some embodiments, the RNAi sequence comprises a sequence complementary to the human RIP3, accession number NP_{—}006862.2, or a fragment thereof. According to certain embodiments the RNAi sequence is complementary to the RIP1 polynucleotide. According to some embodiments, the RNAi sequence comprises a sequence complementary to the human RIP1, accession number NP_{—}003795.2, or a fragment thereof.

In some embodiments gene expression is down-regulated by at least 25%, preferably at least 50%, at least 70%, 80% or at least 90%. In certain other embodiments, partial down-regulation is preferred. Examples for expression-inhibiting (down-regulating or silencing) oligonucleic acids are antisense molecules, RNA interfering molecules (RNAi), and enzymatic nucleic acid molecules, as detailed herein.

“Complementarity” refers to the ability of a nucleic acid to form hydrogen bond(s) with another nucleic acid sequence. A percent complementarity indicates the percentage of contiguous residues in a nucleic acid molecule, which can form hydrogen bonds (e.g., Watson-Crick base pairing) with a second nucleic acid sequence. “Fully complementary” means that all the contiguous residues of a nucleic acid sequence will form a hydrogen bond with the same number of contiguous residues in a second nucleic acid sequence. The term “substantially” complementary as used herein refers to a molecule in which about 80% of the contiguous residues of a nucleic acid sequence will form a hydrogen bond with the same number of contiguous residues in a second nucleic acid sequence. In some embodiments substantially complementary refers to 85%, 90%, 95% of the contiguous residues of a nucleic acid sequence hydrogen bonding with the same number of contiguous residues in a second nucleic acid sequence.

Expression of a given gene can also be inhibited by an enzymatic nucleic acid. As used herein, an “enzymatic nucleic acid” refers to a nucleic acid comprising a substrate binding region that has complementarity to a contiguous nucleic acid sequence of a gene, and which is able to specifically cleave the gene. The enzymatic nucleic acid substrate binding region can be, for example, 50-100% complementary, 75-100% complementary, or 95-100% complementary to a contiguous nucleic acid sequence in a gene. The enzymatic nucleic acids can also comprise modifications at the base, sugar, and/or phosphate groups. An exemplary enzymatic nucleic acid for use in the present methods is a ribozyme. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, and RNAzyme.

The possibility of designing ribozymes to cleave any specific target RNA has rendered them valuable tools in both basic research and therapeutic applications. In therapeutics, ribozymes have been exploited to target viral RNAs in infectious diseases, dominant oncogenes in cancers and specific somatic mutations in genetic disorders. Ribozymes and ribozyme analogs are described, for example, in U.S. Pat. Nos. 5,436,330, 5,545,729 and 5,631,115.

According to yet additional embodiments, the RIP kinase inhibitor is a protein, a polypeptide or a peptide. The protein, polypeptide or peptide may be a synthetic or a recombinant protein, polypeptide or peptide. The protein, polypeptide or peptide may be a chimeric or fusion protein, polypeptide or peptide composed of at least two portions of a protein, polypeptide or peptide.

According to certain embodiments, the RIP inhibitor is capable of passing through the blood brain barrier (BBB). There are several means for delivering compound through BBB as disclosed, for example, in U.S. Pat. Nos. 8,629,114, 8,497,246, and 7,981,864. The RIP inhibitor compounds may be fused or conjugated to BBB transfer compounds as described in the art.

The protein or polypeptide may be conjugated to (covalently) or complexed with (non-covalently) a compound selected from the group consisting of: polyethylene glycol, a copolymer of ethylene glycol, a polypropylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid, a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group, an immunoglobulin light chain and heavy chain, an immunoglobulin Fc domain or portion thereof, a CH2 domain of Fc, an Fc domain loop, an albumin, an albumin-binding protein, a transthyretin and a thyroxine-binding globulin. In exemplary embodiments, said RIP inhibitor polypeptide or derivative is conjugated to or complexed with polyethylene glycol (PEG). In other exemplary embodiments, said RIP inhibitor polypeptide or derivative is conjugated to or complexed with an immunoglobulin Fc domain or portion thereof.

According to yet additional aspect, the present invention provides a pharmaceutical composition comprising as an active ingredient a RIP kinase inhibitor and a pharmaceutically acceptable carrier, excipient or diluent.

According to other embodiments, the present invention provides a kit for the treatment of lysosomal storage disease comprising a pharmaceutical composition comprising at least one RIP inhibitor and carrier; and instruction for use of said pharmaceutical composition for the treatment of lysosomal storage disease.

As used herein a “pharmaceutical composition” refers to a preparation of one or more of the RIP inhibitors described herein and as may be found in the art, with other components such as pharmaceutically acceptable carriers and excipients. The purpose of a pharmaceutical composition is to facilitate administration of a compound to a subject.

As used herein, the term “excipient” refers to an inert substance added to a pharmaceutical composition to further facilitate administration of the RIP inhibitor. Examples, without limitation, of excipients include calcium carbonate, calcium phosphate, various sugars and types of starch, cellulose derivatives, gelatin, vegetable oils, and polyethylene glycols.

As used herein, the phrase “pharmaceutically acceptable carrier” refers to a carrier, an excipient or a diluent that does not cause significant irritation to an organism and does not abrogate the biological activity and properties of the administered compound.

As used herein, the term “carrier” refers to any substance suitable as a vehicle for delivering of the RIP inhibitor of the present invention to a suitable biological site or tissue. As such, carriers can act as a pharmaceutically acceptable excipient of the pharmaceutical composition of the present invention. Carriers of the present invention include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Furthermore, the pharmaceutical composition according to the invention may comprise one or more stabilizers such as, for example, carbohydrates including sorbitol, mannitol, starch, sucrose, dextrin and glucose, proteins such as albumin or casein, and buffers like alkaline phosphates. Pharmaceutical compositions of the present invention may be sterilized by conventional methods.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. A pharmaceutical composition of the invention is formulated to be compatible with its intended route of administration. Methods to accomplish the administration are known to those of ordinary skill in the art. Examples of suitable excipients and modes for formulating the compositions are described in the latest edition of “Remington's Pharmaceutical Sciences” by E. W. Martin.

As a non-limiting example, the pharmaceutical composition of the invention may be administered to a patient afflicted with a lysosomal storage disease who is currently in remission in order to inhibit reinstatement of the disease or to inhibit further progression of the disease. As another non-limiting example, the pharmaceutical composition may be administered to a patient afflicted with a lysosomal storage disease with manifested LSD symptoms in order to reduce the severity of lysosomal storage disease in the subject. As another non-limiting example, the pharmaceutical composition may be administered to a patient affected with a lysosomal storage disease with manifested lysosomal storage disease symptoms in order to ameliorate the symptoms of lysosomal storage disease. As another non-limiting example, the pharmaceutical composition may be administered to a patient affected with a lysosomal storage disease with manifested lysosomal storage disease symptoms in order to inhibit further progression of the lysosomal storage disease. As another non-limiting example, the pharmaceutical composition may be administered to a patient afflicted with lysosomal storage disease with manifested lysosomal storage disease symptoms in order to cure the lysosomal storage disease. As another non-limiting example, the pharmaceutical composition may be administered to a patient diagnosed with lysosomal storage disease in order to prevent the manifestation of lysosomal storage disease symptoms. As another non-limiting example, the pharmaceutical composition may be administered to a patient with Gba mutant/s which found to be susceptible to lysosomal storage disease and/or neurodegenerative diseases. In exemplary embodiments, said neurodegenerative disease is Parkinson.

In certain embodiments, the pharmaceutical composition of the invention may be administered to a patient affected with Krabbe disease.

In certain embodiments of the present invention, the RIP inhibitor(s) therapeutic agent(s) is administered in a dosage form that permits systemic uptake, such that the therapeutic agent(s) may cross the blood-brain barrier so as to exert effects on neuronal cells. For example, pharmaceutical formulations of the therapeutic agent(s) suitable for parenteral/injectable used generally include sterile aqueous solutions (where water soluble), or dispersions and sterile powders for the extemporaneous preparation of sterile injectable solutions or dispersion. In all cases, the form must be sterile and must be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, polyethylene glycol, and the like), suitable mixtures thereof, or vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate or gelatin.

Sterile injectable solutions are prepared by incorporating the therapeutic agent(s) in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof. Pharmaceutical compositions according to the invention are typically liquid formulations suitable for injection or infusion. For example, saline solutions and aqueous dextrose and glycerol solutions can be employed as liquid carriers, particularly for injectable solutions.

Solutions or suspensions used for intravenous administration typically include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin. Where necessary, the composition may also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Oral compositions include an inert diluent or edible carrier. The composition can be enclosed in gelatin or compressed into tablets. For the purpose of oral administration, the active agent can be incorporated with excipients and placed in tablets, troches, or capsules. Pharmaceutically compatible binding agents or adjuvant materials can be included in the composition. The tablets, troches, and capsules. may optionally contain a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; or a sweetening agent or a flavoring agent.

The composition may also be administered by a transmucosal or transdermal route. Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. The composition can be formulated as a suppository, with traditional binders and carriers such as triglycerides.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

In certain embodiments, polypeptide active agents are prepared with carriers to protect the polypeptide against rapid elimination from the body. Biodegradable polymers (e.g., ethylene vinyl acetate, polyanhydrides, polyglycolic acid, collagen, polyorthoesters, and polylactic acid) are often used. Methods for the preparation of such formulations are known by those skilled in the art. Liposomal suspensions can be used as pharmaceutically acceptable carriers too. The liposomes can be prepared according to established methods known in the art (for example, U.S. Pat. No. 4,522,811).

“Targeting carriers” are interchangeable with “delivery vehicles”. Delivery vehicles according to the present invention include agents that are capable of delivering the RIP inhibitor of the present invention to a target site in a subject. A “target site” refers to a site in a subject to which one desires to deliver the RIP inhibitor. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of the RIP inhibitor of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type or tissue. “Specifically targeting” refers to causing a delivery vehicle to bind to a particular cell by the interaction of the compound in the vehicle to a molecule on the surface of the cell. Suitable “targeting compounds” include ligands capable of selectively (i.e., specifically) binding another molecule at a particular site. Examples of such ligands include antibodies, antigens, receptors and receptor ligands. For example, an antibody specific for an antigen found on the surface of a target cell can be introduced to the outer surface of a liposome delivery vehicle so as to target the delivery vehicle to the target cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Alternatively, the RIP inhibitor of the present invention may be targeted to a specific tissue within the body by modifying the RIP inhibitor, such that it would be bound to the “targeting compounds” defined hereinabove.

According to yet additional embodiments, the composition comprising a RIP inhibitor may further comprise an additional agent capable of treating lysosomal storage disease.

The term “subject” includes humans and animals afflicted with LSD and human or animals amenable to therapy with a RIP inhibitor. According to yet another embodiment, the subject is a human.

The term “subject” as used herein, includes, for example, a subject who has been diagnosed to be afflicted with LSD or a subject who has been treated to ameliorate LSD, including subjects that have been refractory to previous treatments of the disease.

The RIP inhibitor of the present invention may be administered systemically or locally. Non limiting examples of systemic routes include, but are not limited to, intravenous, intraarterial, intraperitoneal, and subcutaneous routes.

The administered dose of the RIP inhibitor in the method of the present invention may be determined while taking into consideration various conditions of a subject that requires treatment, for example, the severity of symptoms, general health conditions of the subject, age, weight, sex of the subject, diet, the timing and frequency of administration, a medicine used in combination, responsiveness to treatment, and compliance with treatment.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Animals and Brain Tissues

Gba^{flox/flox}; mice were crossed with Gba^{flox/+}; Nestin-Cre mice to generate Gba^{flox/flox}; Nestin-Cre mice (Enquist, I. B. et al. 2007. Proc. Natl. Acad. Sci. U.S.A. 104, 17483-17488) and Gba^{flox/+}; Nestin-Cre mice, which served as healthy controls. Genotyping was performed by PCR using genomic DNA extracted from mouse tails or embryonic brains (Farfel-Becker, T. et al. 2009. Hum. Mol. Genet. 18, 1482-1488). Both male and female mice were used. The colony was maintained in the experimental animal center of the Weizmann Institute of Science. All animal experiments were approved by the Weizmann Institute Institutional Animal Care and Use Committee. Mice deficient in galactocerebrosidase (the Twitcher mouse) were used as a model of Krabbe disease (Suzuki. K. & Taniike, M. 1995. Res. Tech. 32, 204-214), mice deficient in the β-subunit of β-hexosaminidase A and B were used as a model of Sandhoff disease (Sango, K. et al. 1995. Nat. Genet. 11, 170-176). Brains from a mouse model of the GM1 gangliosidosis (Hahn, C. N. et al. 1997. Hum. Mol. Genet. 6, 205-211), defective in lysosomal β-galactosidase, and from Niemann-Pick disease Type C1 mice (Pentchev, P. G. et al. 1984. J. Biol. Chem. 259, 5784-5791), defective in the NPC1 gene, were also used. Mice deficient in TNFα (strain B6; 129S6-Tnftm1Gkl/J, The Jackson Laboratory) were provided by Dr. David Wallach (Weizmann Institute, Israel). C57BL/6J (The Jackson Laboratory) mice were injected daily intraperitoneally with 50 mg CBE/kg body weight/day (Kanfer, J. N. et al. 1975. Biochem. Biophys. Res. Commun. 67, 85-90) or with phosphate-buffered saline from 8 days of age.

### Rip3 Null Mice

Rp3−/− mice (Newton, K., Sun, X. & Dixit, V. M. 2004. Cell. Biol. 24, 1464-1469) were provided by Genentech (South San Francisco, Calif.) and backcrossed with C57BL/6 mice to generate Rip3+/− mice. Rip3+/− mice were crossed with Rip3−/− mice to generate Rip3+/− and Rip3−/− littermates. Rip3+/− and Rip3−/− mice were injected daily intraperitoneally with 25 mg CBE/kg body weight/day (Kanfer et al. 1975, ibid) or with phosphate-buffered saline from 8 days of age. No animals were excluded from the study; the sample size was chosen so as to validate statistical analyses. No randomization was used and the investigator was not blinded.

### Histochemistry

Tissue was prepared as described, in Vinter et al. 2010 (Vitner, E. B. et al. 2010. Hum. Mol. Genet. 19, 3583-3590). Paraffin sections were incubated with anti-RIP3 (1:100, ProSci, 2283) (Narayan, N. et al. 2012. Nature 492, 199-204). Anti-NeuN (1:50, Chemicon, MAB377) (Vitner, E. B., et al. 2012. Brain 135. 1724-1735), anti-Mac2 (microglial activation marker, 1:250, Cedarlane, CL8942AP) (Farfel-Becker, T. 2011 ibid) and anti-GFAP (1:100, Dako, 20334) (Farfel-Becker et al. 2011, ibid) antibodies. Counterstaining was performed with 4′,6-diamidino-2-phenylindole (DAPI) (Molecular Probes, Eugene, Oreg., USA) (1:2000 dilution, 5 min). Apoptotic cells were detected using TUNEL (ApopTag Red in situ kit, Chemicon, Temecula, Calif.). Kuppfer cells were stained with a rat anti-CD68 antibody (1:1000, Serotec, MCA1957) (Lopez, M. E., et at 2011. J. Neurosci. 31, 4367-4378) on floating sections. Degenerating neurons were stained with the anionic fluorescein derivative, FluoroJade C, according to manufacturer's instructions (HistoChem, Jefferson, Ark.).

### Caspase Activity

Caspases 3/7, 8, and 9 were assayed using a Caspase-Glo assay kit (Promega).

RNA Extraction and qPCR

Cortical tissues were used for total RNA extraction. Total RNA isolation, cDNA synthesis and real-time quantitative PCR were performed as described in Vinter et al. 2010 (ibid). The relative amounts of mRNA were calculated from the cycle threshold (CT) values using TBP for normalization. qPCR was performed using the SYBR Green methods with the following primers:

TBP forward:
(SEQ ID NO: 1)
5′-TGCTGTTGGTGATTGTTGGT-3′;
TBP reverse:
(SEQ ID NO: 2)
5′-CTGGCTTGTGTGGGAAAGAT-3′;
RIP1 forward:
(SEQ ID NO: 3)
5′-AGTCGAGACTGAAGGACACAGCACT-3′;
RIP1 reverse:
(SEQ ID NO: 4)
5′-TCCAGCAGGTCACTGGATGCCAT-3′;
RIP3 forward:
(SEQ ID NO: 5)
5′-CTTGAACCCTCCGCTCCTGC-3′;
RIP3 reverse:
(SEQ ID NO: 6)
5′-AATCTGCTAGCTTGGCGTGG-3′;
_{C}FLIP_{L} forward:
(SEQ ID NO: 7)
5′-ACATGTGTGCTCTGTGGAGG-3′;
_{C}FLIP_{L} reverse:
(SEQ ID NO: 8)
5′-TGCCTGGCTGATTCTGTCTC-3′;
_{C}FLIP_{R} forward:
(SEQ ID NO: 9)
5′-ACCTCACGGAACTCATGTCC-3′;
_{C}FLIP_{R} reverse:
(SEQ ID NO: 10)
5′-TGGGTAGATTCTCTGTGCATGG-3′.

p values were calculated using a one-tailed two-independent samples Student's t test. A p value≦0.05 was considered statistically significant.

### Protein Extraction and Western Blotting

Homogenates were prepared as described in Vinter et al. 2010 (ibid). Blots were incubated with the following antibodies: rabbit anti-RIP1 (van Raam, B. J. et al. 2013. Cell Death Differ. 20, 86-96), (1:1000, Cell signaling, 3493), rabbit anti-cleaved PARP (Hung, T. et al. 2011. Nat. Genet. 43, 621-629) (1:1000, Cell Signaling, 9544), rabbit anti-RIP3 (Narayan et al. 2012, ibid) (1:1000, ProSci, 2283), Rat anti-caspase 8 (Kovalenko, A. et al. 2009. J. Exp. Med. 206, 2161-2177), (1:2000. Enzo Biochem, ALX-804-448), rabbit anti-FLIP (Han, J. et al. 2013. Mol. Biol. Cell 24, 465-473) (1:1000, Cell signaling, 3210) and anti-GAPDH (Vinter et al. 2012, ibid) (1:10000, Chemicon, MAB374), prior to incubation with a horseradish peroxidase (HRP)-conjugated secondary antibody (Jackson ImmunoResearch). Bound antibodies were detected using the SuperSignal West Pico chemiluminescent substrate (Thermo Scientific).

### Serum Alanine Aminotransferase (ALT)

ALT was detected using Spotchem II strips (Arkray, Japan).

### Behavioral Testing

A Rotarod test (Harvard equipment) was used to evaluate Rotarod behavior in 3 and 4 week-old mice using an accelerating paradigm (4 min on the Rotarod at 40 r.p.m.).

### Statistical Analyses

All data are shown as the average±s.e.m. Comparisons between two samples were performed using a two-tailed Student's t test. P<0.05 was considered statistically significant.

### Example 1

### Neuronal Cell Death in Neuropathic Gaucher Disease (nGD) is Caspase-Independent and Non-Apoptotic

Two independent GD models were used to determine the mechanism of neuronal cell death in nGD: the genetic Gba^{flox/flox}; Nestin-Cre mice and a chemically-induced model in which the irreversible GlcCerase inhibitor, conduritol β-epoxide (CBE), is injected intra-peritoneally daily to mice (Kanfer et al. 1975. ibid). Gba^{flox/flox} mice were crossed with Gba^{flox/+}; nestin-Cre mice to generate Gba^{flox/flox}; nestin-Cre mice (referred to as −/− nGD mice), and Gba^{flox/+}; nestin-Cre mice (referred to as +/− nGD mice), which served as healthy controls. These mice exhibit rapid motor dysfunction including rigidity of limbs and abnormal gait. leading to seizures and paralysis by 21 days of age, at which time mice exhibit massive microglial activation, astrocytosis and neuron loss. Some of the inventors of the present invention observed previously severe neuronal loss in brain areas of nGD mice. Specifically, Niss1 staining which provides estimate of the neuronal cell number demonstrated of progressive neuronal loss in the −/− nGD mice (Farfel-Becker et al., 2011, ibid).

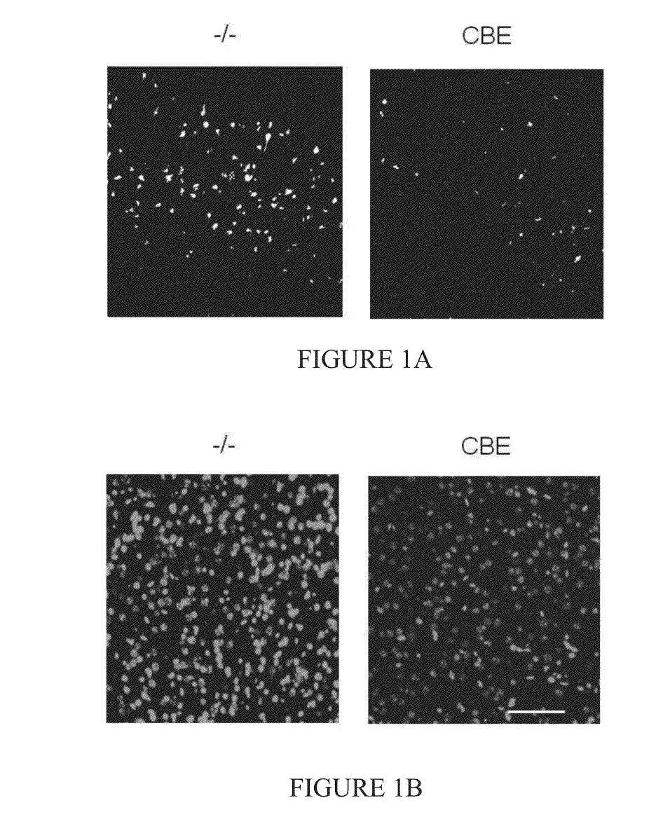

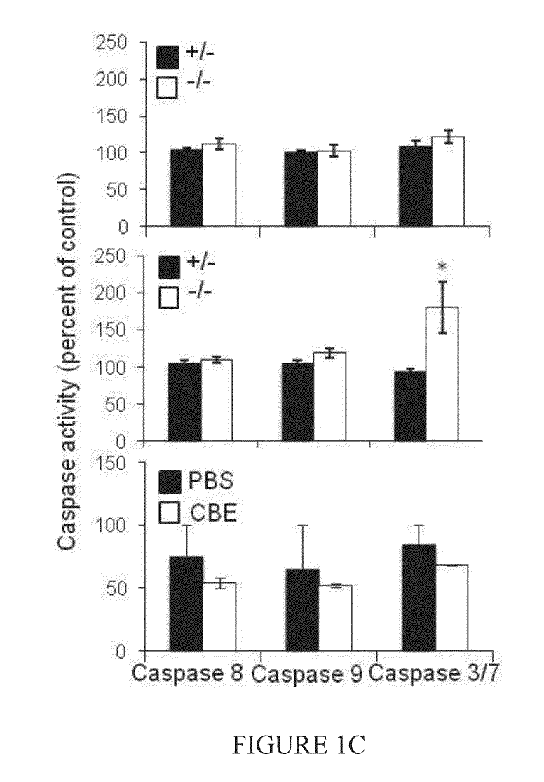

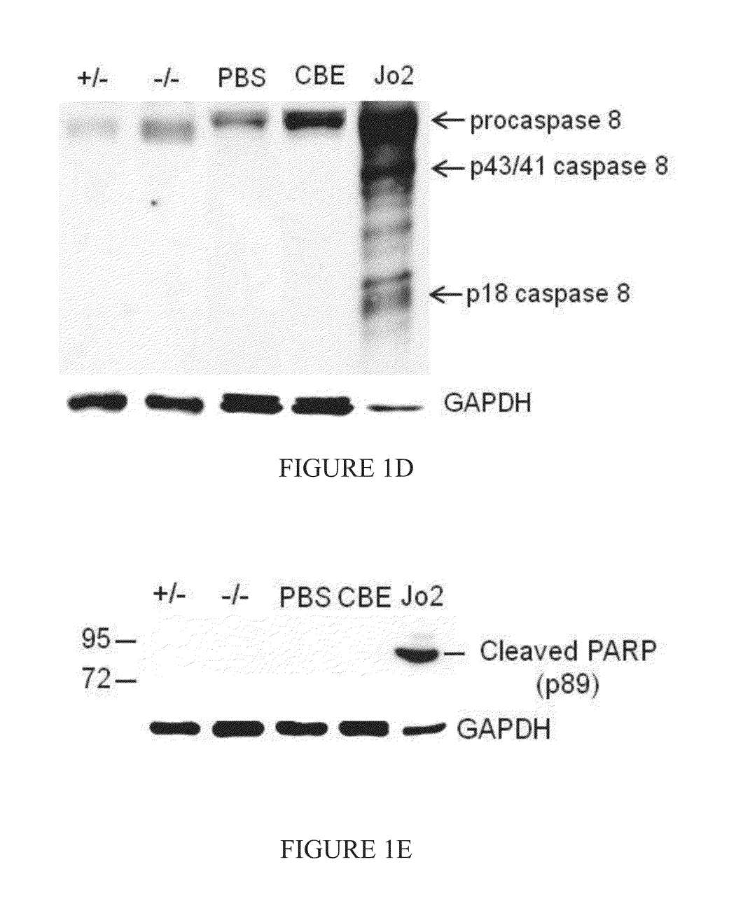

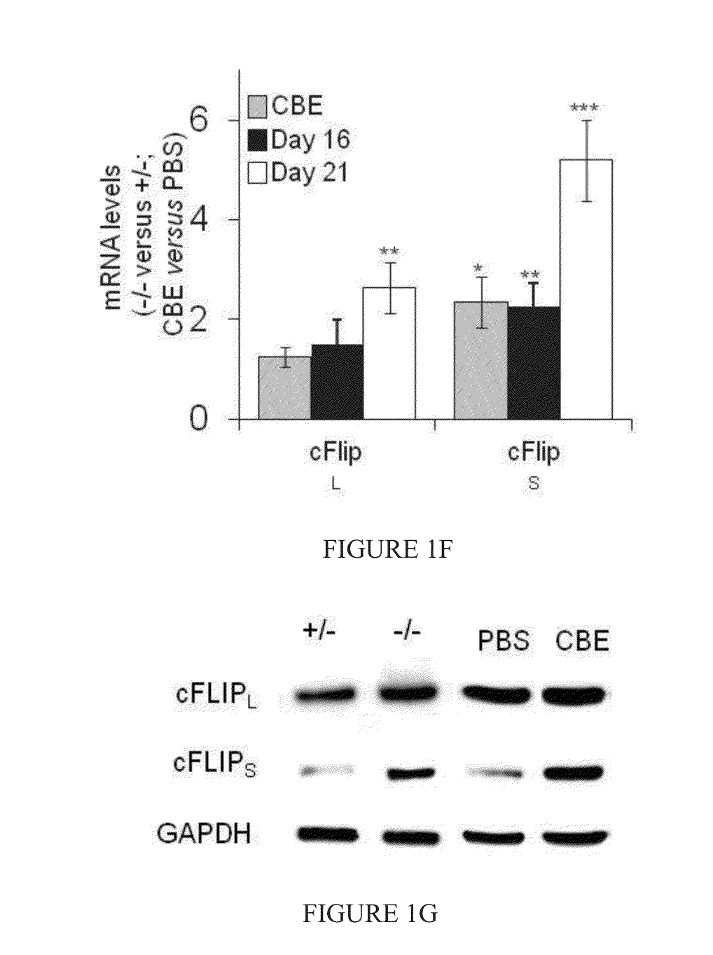

Although both Niss1 and FluoroJade C staining (FIG. 1A) detected profound levels of cell death in the cerebral cortex of 16 and 21 day-old Gba^{flox/flox}; Nestin-Cre mice and in 27-day old CBE-treated mice, no TUNEL-positive cells were observed in either model (FIG. 1B; only DAPI staining has been detected). Similarly, there was no elevation in the activities of caspases 9 and 3/7 (FIG. 1C), with the exception of a modest elevation in caspase 3/7 at the terminal disease stage in Gba^{flox/flox}; Nestin-Cre mice and of caspase 8 (FIG. 1C). In addition, no cleavage of caspase 8 (FIG. 1D) or of PARP (poly (ADP-ribose) polymerase) (FIG. 1E) have been observed, suggesting that neuronal cell death in nGD is caspase-independent and non-apoptotic.

In both GD mouse models, elevated levels of cFLIPS were detected in the brains of symptomatic mice (by mRNA levels, FIG. 1F, and by Western blotting, FIG. 1G). These results suggest the presence of a caspase 8/cFLIPS heterodimer, which would explain the lack of caspase 8 activity (FIG. 1C) in nGD mouse brain. Elevation in cFLIPL mRNA levels was only detected at the terminal stage of the disease in Gba^{flox/flox}; Nestin-Cre mice (FIG. 1F).

### Example 2

### Elevation of Receptor-Interacting Protein (RIPs) Kinases in nGD Brains

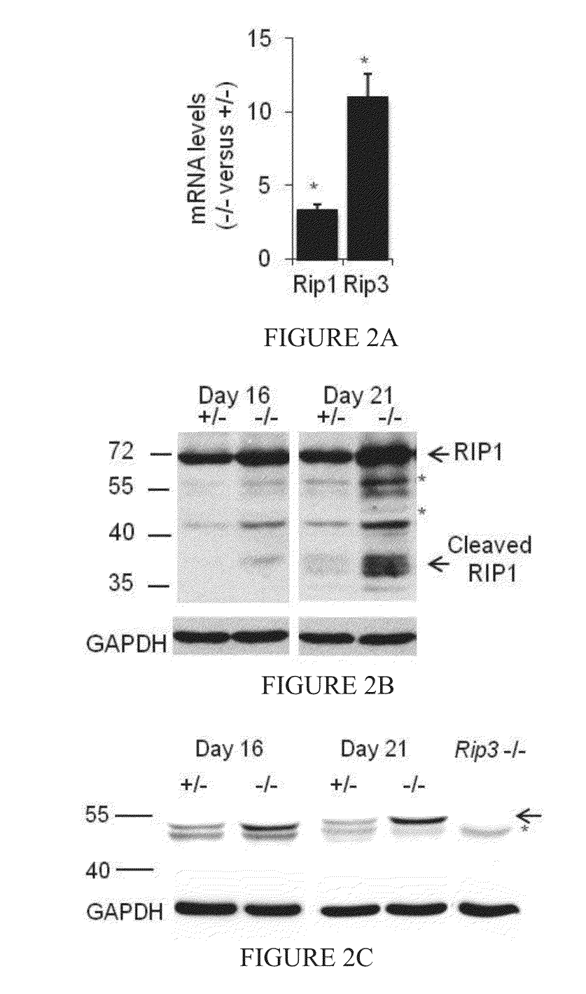

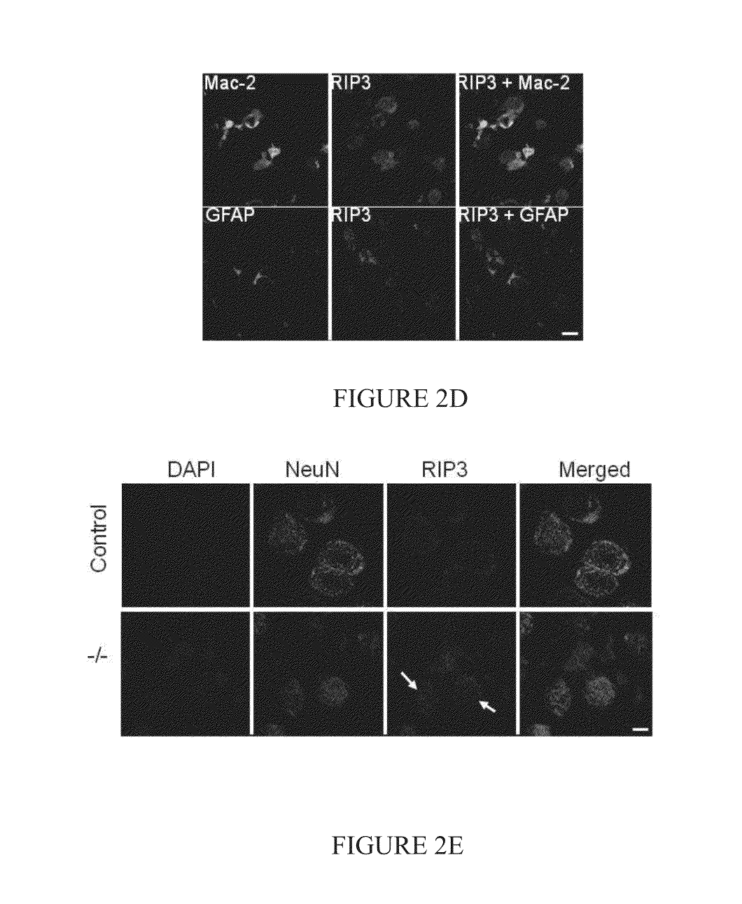

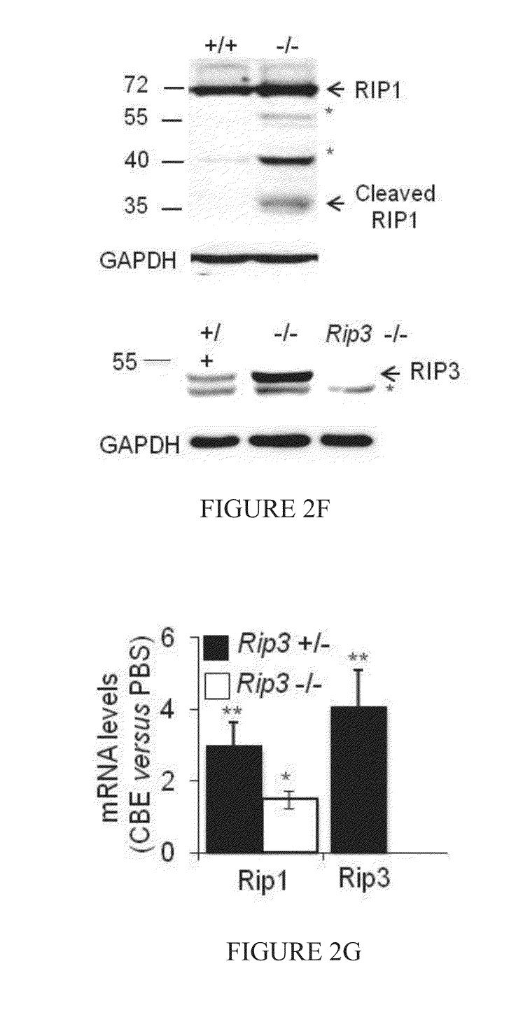

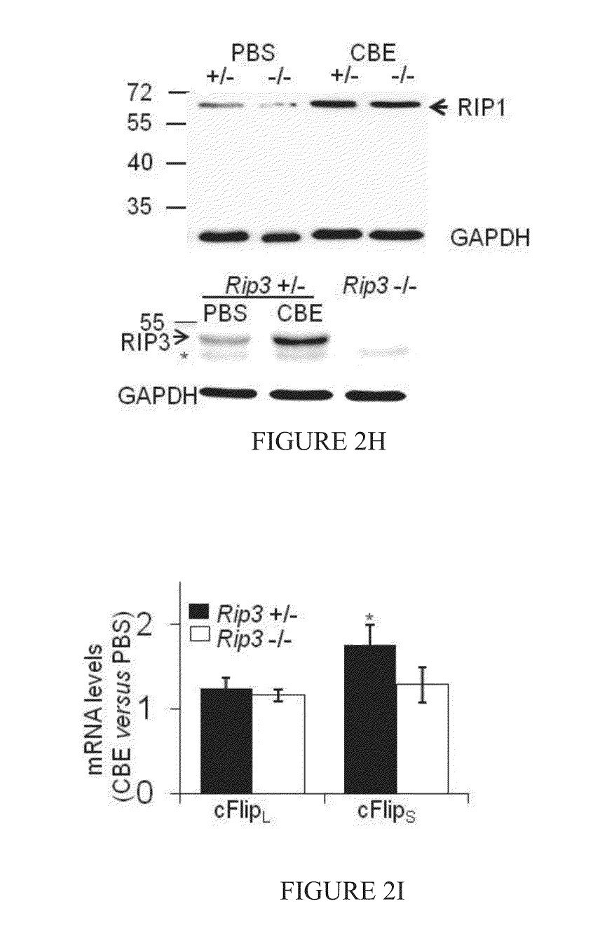

Increased expression of RIP1 and RIP3, and their contribution to various pathological conditions have been reported, including detachment of the retina, macrophage necrosis in atherosclerosis development, regulation of virus-induced inflammation, systemic inflammatory response syndrome and ethanol-induced liver injury. All of these pathological states exhibit necrotic cell death and the involvement of RIP1 and RIP3 has been attributed to their role in necrosis. To elucidate the role of necroptosis in nGD brain, the levels of RIP1 and RIP3 were analyzed. As is shown in FIG. 2, expression of both genes was markedly elevated, as determined by analysis of mRNA levels (FIG. 2A) and Western blotting (FIG. 2B, C) in the brains of symptomatic Gba^{flox/flox}; Nestin-Cre mice. Crucially, levels of RIP1 were also elevated in the one available brain of a human patient who succumbed to type 2 GD (data not shown).

An additional role of RIP1 and RIP3 in the inflammatory processes has recently been suggested (Kang, T.-B. et al. 2013. Immunity 38, 27-40); RIP1 and RIP3 can contribute to inflammation, independent of cell death, by activating the NLRP3 inflammasome, a signaling complex that, through activation of caspase 1, mediates processing of the precursors for proinflammatory mediators such as IL-1β, in myeloid cells. The role of RIP3 in pro-inflammatory processes is also supported by the fact that epidermis specific elimination of caspase 8 leads to chronic inflammation that can be suppressed by deletion of RIP3. To elucidate the role of RIP3 in nGD brain, it was analyzed whether RIP3 abundance was increased in microglia, in neurons or in astrocytes. In Gba^{flox/flox}; Nestin-Cre mouse brain. RIP3 was increased in all Mac-2 positive (i.e. activated) microglia (FIG. 2D), consistent with a neuroinflammatory role of RIP3. RIP3 was principally expressed in the nuclei of neurons from Gba^{flox/flox}; Nestin-Cre mice, in striking contrast to control mice where it was located in the neuronal cytoplasm (FIG. 2E), implying a possible role in neuronal cell death. Translocation of RIP3 to the nucleus has been observed in HeLa cells upon treatment with leptomycin B29, but RIP3 translocation in the CNS has not been reported. However, RIP3 was undetectable in GFAP-positive astrocytes (FIG. 2D).

### Example 3

### RIPs Expression in Other Lysosomal Storage Disorders (LSDs)

A direct correlation was observed between the presence of the immunoreactive RIP3 signal and the brain regions that are affected in nGD (Farfel-Becker et al. 2011, ibid). Notably, RIP1 and RIP3 were unaltered in brains obtained from murine strains that authentically model other LSDs, such as Niemann Pick type C1, GM1 gangliosidosis and Sandhoff disease (data not shown). However, RIP1 and RIP3 expression was strikingly elevated (˜5-fold and ˜3-fold, respectively) in the brains of Twitcher mice, which lack β-galactocerebrosidase and act as an authentic murine model of Krabbe disease (FIG. 2F). Not only does Krabbe disease resemble nGD in as much as it causes acute neurodegeneration in infants, but it is also caused by the inability to hydrolyze a simple mono-glycosylated glycosphingolipid (galactosylceramide in the case of Krabbe disease and glucosylceramide in case of GD). Moreover, infiltration of the CNS by multinucleated giant cells of macrophage lineage (known as Gaucher cells and globoid cells in Krabbe and GD, respectively) is unique to these glycosphingolipidoses; in addition, the cognate deacylated metabolites, 1-β-glucosyl- and 1-β-galactosylsphingosine (‘psychosine’) have been implicated in the neuropathology of both diseases. Together, these data indicate that although brain inflammation and microglial activation are shared features of many LSDs, different pathways of neuroinflammation occur in specific LSDs.

### Example 4

### RIP3 Deficiency Improves the Clinical Manifestation of nGD Mice

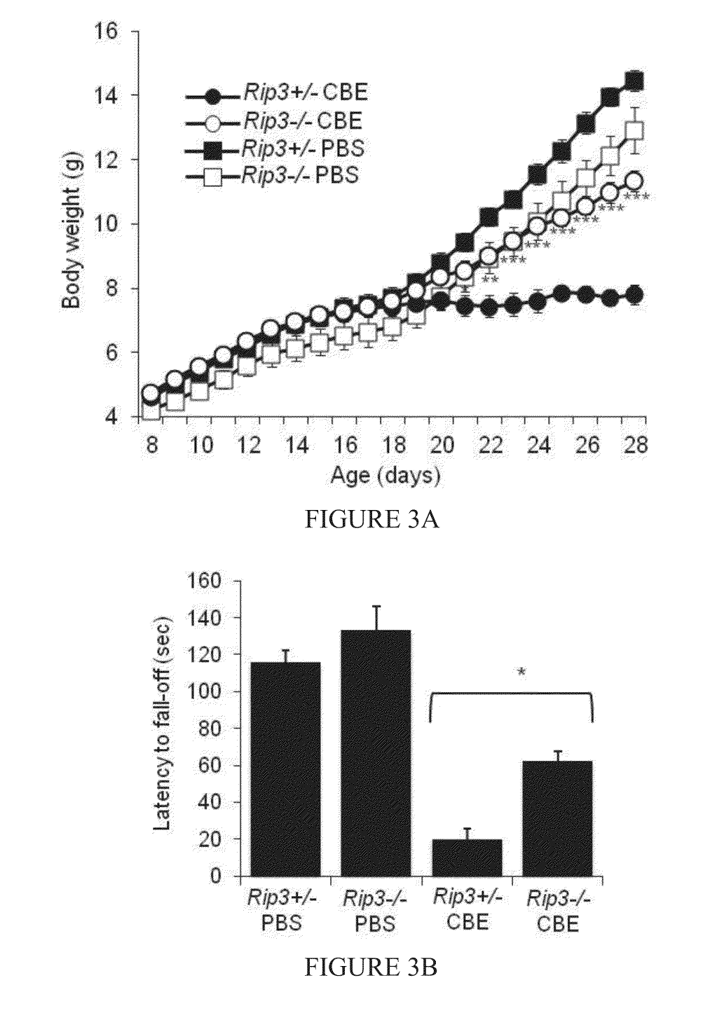

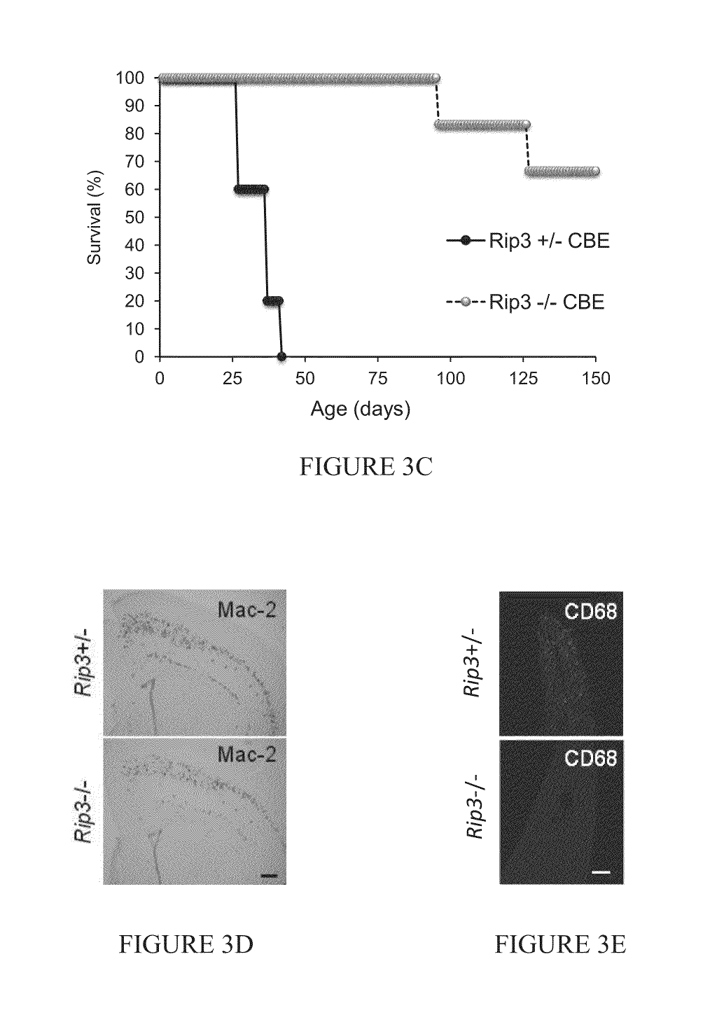

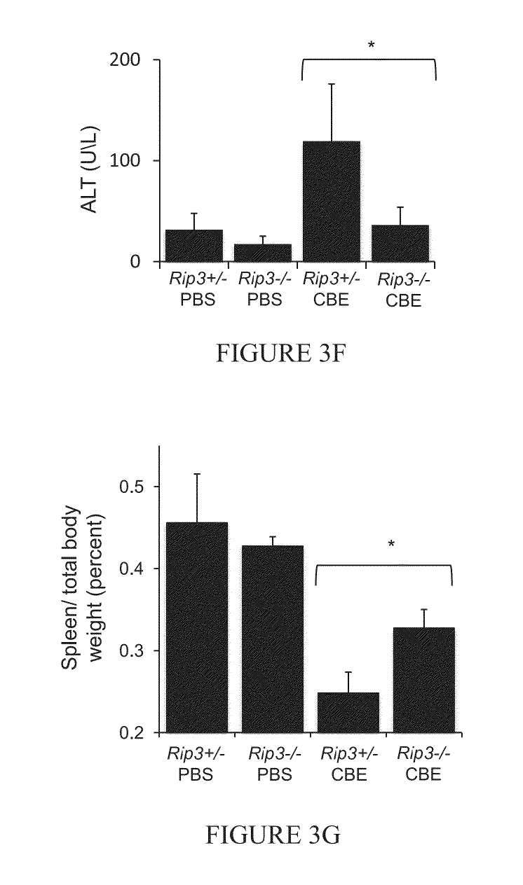

To analyze further the role of RIP in nGD pathology, GD was induced in Rip3-deficient mice. In contrast to Rip1 null mice which die 1-3 days after birth (Kelliher, M. A. et al. 1998. Immunity 8, 297-303), thus rendering them unsuitable for in vivo studies, Rip3 deficiency has no demonstrable adverse effect on mouse development or health. GD was induced by daily injections of CBE. The CBE model was chosen because GlcCerase activity is inhibited in all cell types and organs upon CBE treatment, in contrast to the Gba^{flox/flox}; Nestin-Cre mouse in which GlcCerase deficiency is restricted to cells of neuronal lineage. Moreover, the latter model is very severe, with mice not surviving beyond three to four weeks of age, limiting the available window of therapeutic intervention. Similar to observations in the Gba^{flox/flox}; Nestin-Cre mouse, levels of RIP1, RIP3 (determined by analysis of mRNA levels (FIG. 2G) and by Western blotting (FIG. 2H)) and mRNA levels of cFLIPS (FIG. 2I) were markedly elevated in the brains of Rip3+/− CBE-treated mice. Remarkably, whereas Rip3+/− mice (control mice) injected with CBE displayed typical manifestations of murine GD (i.e. weight loss (FIG. 3A) and loss of motor coordination (FIG. 3B)), the signs of disease in Rip3−/− mice injected with CBE were dramatically ameliorated (FIG. 3A, B). Notably, the life-span of Rip3−/− mice injected with CBE was significantly extended to >100 days, with survival to 180 days in some animals, whereas no Rip3+/− mice survived beyond 40 days of age (FIG. 3C). Importantly, the improvements in motor coordination (FIG. 3B) and life-span (FIG. 3C) were observed prior to the appearance of neuronal loss, but after the appearance of neuroinflammation, and were accompanied by markedly fewer activated microglia in layer V of the cortex (FIG. 3D). These results directly implicate the RIP3 kinase pathway in neuroinflammation, and indicate that this pathway might be a molecular target for therapeutic intervention in nGD. Moreover, liver injury was also improved in CBE-treated Rip3−/− mice, which showed fewer CD68-positive Kupffer cells (FIG. 3E) and a decrease in serum alanine aminotransferase (ALT) activity, suggesting that CBE-induced hepatocyte injury was also attenuated (FIG. 3F). Finally, spleen weight, an indicator of GD progression, was also improved (FIG. 3G). Improvement in the outcome of the visceral symptoms, as well as in CNS pathology after CBE administration, supports the hypothesis that RIP3 is critical for a specific mode of microglia/macrophage activation in the inflammatory response.

### Example 5

### Experimentally-Induced GD Pathology is TNFα-Independent

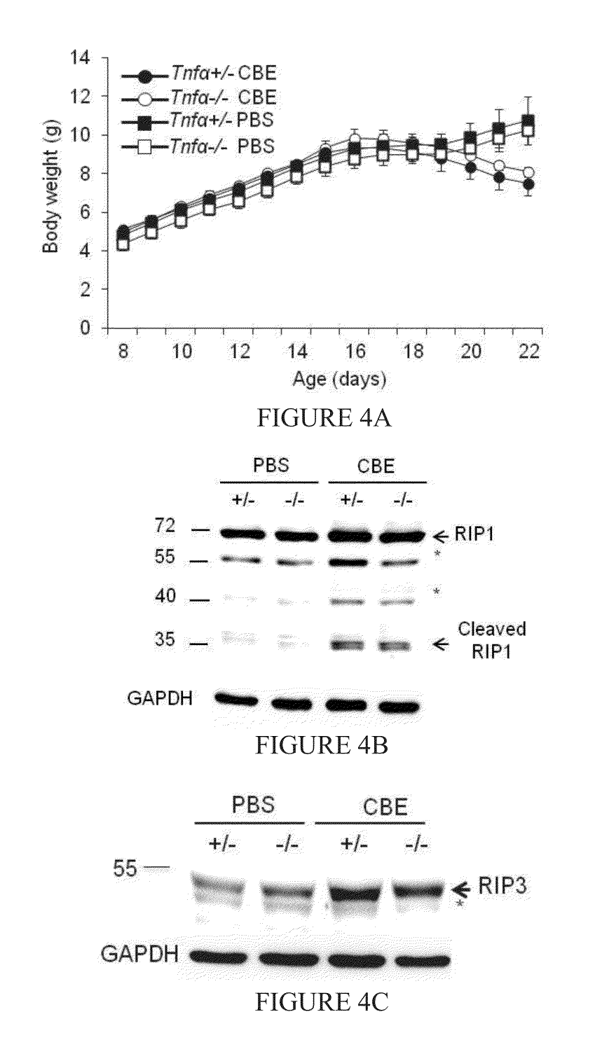

No phenotypic difference in the disease induced by CBE was observed in TNFα-deficient mice (FIG. 4A), and disease progression in these mice was accompanied by elevated RIP1 (FIG. 4B) and RIP3 (FIG. 4C) levels, suggesting that the experimentally-induced GD pathology is RIP3-dependent but TNFα-independent.

### Example 6

### Elevation of Pro Caspase-1 and Pro IL-1β in Brains from Neuropathic Gaucher Disease Mice

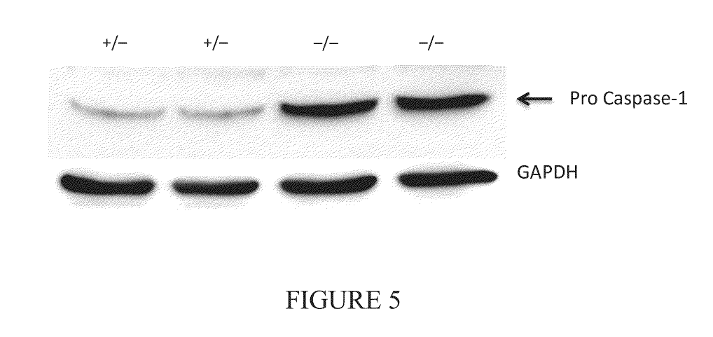

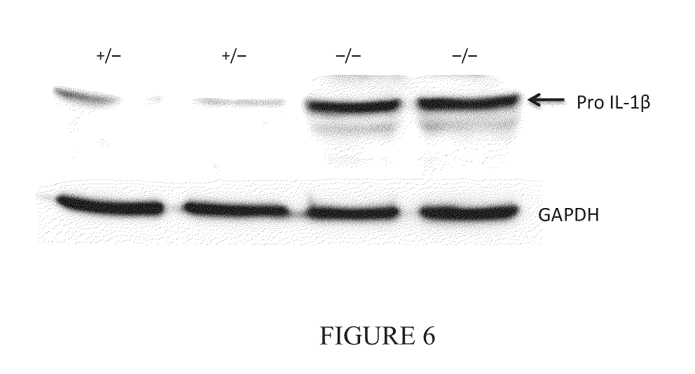

To analyze further the role of additional members of IL-10 inflammation pathway, the expression of Pro caspase-1 and Pro IL-1β was analyzed. Western blotting of homogenates (150 μg of protein) from the brains of 21 day-old Gba^{flox/flox}; Nestin-Cre (−/−) mice with their respective littermate controls (+/−) showed elevated expression of Pro caspase-1 (FIG. 5) and Pro IL-1β (FIG. 6) in the neuropathic GD mice.

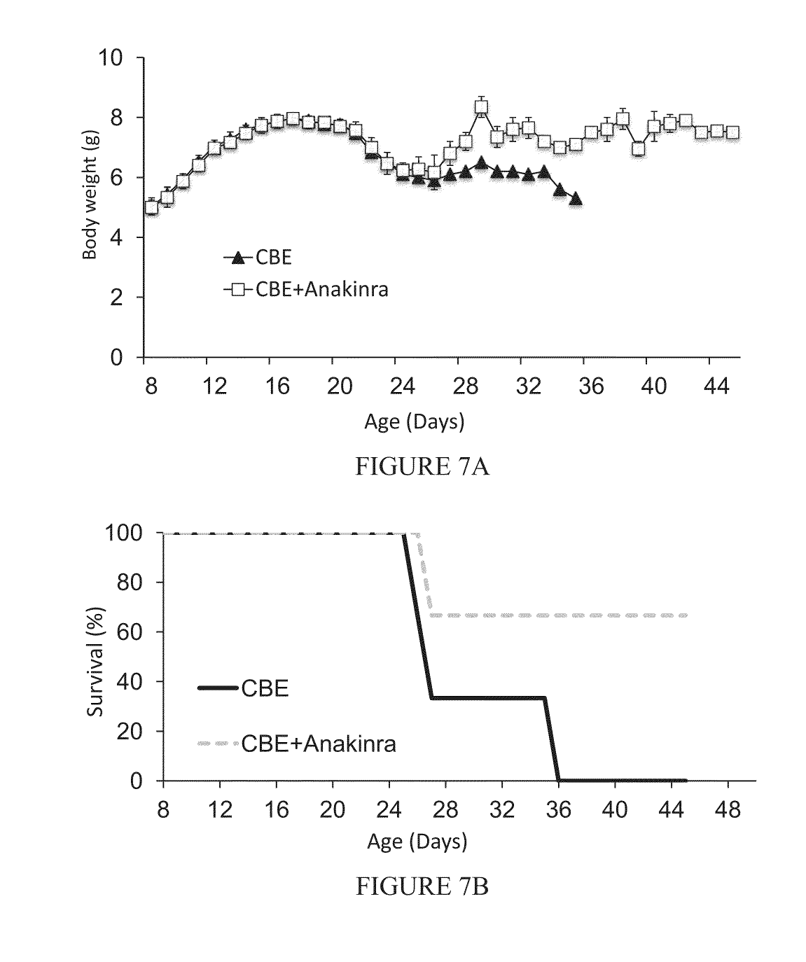

Moreover, treatment of GD mice with IL-1β receptor (Anakinra) improves gain weight (FIG. 7A) and life-span (FIG. 7B) of CBE-treated mice, suggesting the involvement of the inflammasome and IL-1β in GD pathology.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention

1-9. (canceled)

10. A method of treating a subject affected with a lysosomal storage disease characterized by elevation of RIP kinase, the method comprising administering to the subject a therapeutically effective amount of at least one inhibitor of at least one Receptor-Interacting Protein (RIP) kinase, thereby treating the lysosomal storage disease.

11. The method of claim 10, wherein the lysosomal storage disease is selected from the group consisting of Gaucher and Krabbe.

12. The method of claim 10, wherein the lysosomal storage disease is Gaucher.

13. The method of claim 12, wherein said Gaucher disease is selected from the group consisting of: type 1 Gaucher's disease, type 2 Gaucher's disease and type 3 Gaucher's disease.

14-17. (canceled)

18. The method of claim 10, wherein the at least one RIP kinase inhibitor is capable of inhibiting the RIP kinase activity or expression.

19. The method of claim 10, wherein the at least one RIP kinase inhibitor is a small molecule capable of inhibiting said RIP kinase activity.

20. The method of claim 10, wherein the RIP kinase inhibitor is in a form capable of passing the blood brain barrier.

21. The method of claim 10, wherein said RIP kinase is selected from RIP1 and RIP3.

22. The method of claim 21, wherein said RIP is RIP3.

23. The method of claim 10, wherein said subject is a human.

24. The method of claim 10, wherein said method further comprises administering to the subject an effective amount of at least one additional therapeutically active compound.

25. The method of claim 24, wherein the therapeutically active compound is for treating a lysosomal storage disease.

26. The method of claim 25, wherein the lysosomal storage disease is selected from the group consisting of Gaucher's disease and Krabbe disease.

27. The method of claim 24, wherein the therapeutically active compound is IL-1 receptor antagonist.

28. The method of claim 27, wherein the IL-1 receptor antagonist is Anakinra.

29. The method of claim 10, wherein the RIP kinase inhibitor is administered orally or parenterally.

30. The method of claim 29, wherein the RIP kinase inhibitor is administered via a route of administration selected from the group consisting of: intravenously, subcutaneously, intra-arterially, intraperitoneally, ophthalmically, intramuscularly, buccally, rectally, vaginally, intraorbitally, intracerebrally, intradermally, intracranially, intraspinally, intraventricularly, intrathecally, intracisternally, intracapsularly, intrapulmonarily, intranasally, transmucosally, transdermally, inhalation, and any combination thereof.

31-35. (canceled)

36. A kit for the treatment of Lysosomal storage disease characterized by RIP kinase elevation comprising a pharmaceutical composition comprising at least one RIP kinase inhibitor and a pharmaceutically acceptable carrier, excipient or diluent; the kit further comprising at least one additional therapeutically active compound.

37. The kit of claim 36, wherein the Lysosomal storage disease is Gaucher's disease.

38. The kit of claim 36, wherein the Lysosomal storage disease is Krabbe's disease.


SEQUENCE LISTING (10 sequences)

SEQ ID NO: 1 - DNA, 21 bp, Artificial
  Synthetic oligo
tgctgttggtgattgttggtv

SEQ ID NO: 2 - DNA, 20 bp, Artificial
  Synthetic oligo
ctggcttgtgtgggaaagat

SEQ ID NO: 3 - DNA, 25 bp, Artificial
  Synthetic oligo
agtcgagactgaaggacacagcact

SEQ ID NO: 4 - DNA, 23 bp, Artificial
  Synthetic oligo
tccagcaggtcactggatgccat

SEQ ID NO: 5 - DNA, 20 bp, Artificial
  Syntetic oligo
cttgaaccctccgctcctgc

SEQ ID NO: 6 - DNA, 20 bp, Artificial
  Synthetic oligo
aatctgctagcttggcgtgg

SEQ ID NO: 7 - DNA, 20 bp, Artificial
  Synthetic oligo
acatgtgtgctctgtggagg

SEQ ID NO: 8 - DNA, 20 bp, Artificial
  Synthetic oligo
tgcctggctgattctgtctc

SEQ ID NO: 9 - DNA, 20 bp, Artificial
  Synthetic oligo
acctcacggaactcatgtcc

SEQ ID NO: 10 - DNA, 22 bp, Artificial
  Synthetic oligo
tgggtagattctctgtgcatgg